(19) United States
(12) Patent Application Publication
(10) Pub. No.: US 2012/0289917 A1
(43) Pub. Date: Nov. 15, 2012

(54) ENHANCED PERSONAL CARE ABSORBENT ARTICLES

(76) Inventors: Frank P. Abuto, Johns Creek, GA (US); Deborah J. Calewarts, Appleton, WI (US); Jenny L. Day, Woodstock, GA (US); Keyur M. Desai, Alpharetta, GA (US); Jeffrey F. Jurena, Appleton, WI (US); Jian Qin, Appleton, WI (US); Donald E. Waldroup, Roswell, GA (US)

(21) Appl. No.: 13/481,366

(22) Filed: May 25, 2012

Related U.S. Application Data
(63) Continuation-in-part of application No. 13/330,440, filed on Dec. 19, 2011.
Application No. 13/330,440 is a continuation-in-part of application No. 12/979,852, filed on Dec. 28, 2010.

(51) Int. Cl. A61L 15/44; A61L 15/26; A61L 15/46

(52) U.S. Cl. 604/359; 604/367; 604/360

(57) ABSTRACT

The present invention relates to a personal care absorbent article comprising at least two substrates each having an internal and external surface, wherein at least one substrate is a fluid permeable bodyside substrate selected from spunbond, meltblown, coform, airlaid, bonded-carded web, spunlace materials and combinations thereof; at least one substrate is an impermeable backsheet; and an absorbent core disposed in between said substrates; wherein at least the external surface of at least one substrate has applied to it a benefit agent selected from an additive composition wherein said additive composition is a polymer dispersion selected from polyolefin dispersions, polyisoprene dispersions, polybutadiene-styrene block copolymer dispersions, latex dispersions, polyvinyl pyrrolidone-styrene copolymer dispersions, polyvinyl alcohol-ethylene copolymer dispersions, and combinations thereof; an enhancement component selected from microparticles, expandable microspheres, fibers, additional polymer dispersions, scents, anti-bacterials, moisturizers, medicaments, soothers and combinations thereof; and combinations thereof.

### CROSS REFERENCE TO RELATED APPLICATION

This application is a continuation-in-part of U.S. application Ser. No. 13/330,440 filed Dec. 19, 2011, which is a continuation-in-part of U.S. application Ser. No. 12/979,852 filed Dec. 28, 2010.

### FIELD OF THE INVENTION

This invention relates to personal care absorbent articles with an efficient layering system having an advanced polymer composition applied thereon. Particularly, the present invention may embody absorbent articles such as feminine pads, adult incontinence articles, diapers, training pants and the like and eliminates the need for a traditional nonwoven or film bodyside topsheet due to a polymer technology that not only provides advanced protection but also enhances the feel, texture, aesthetics and comfort of the overall article.

### BACKGROUND OF THE INVENTION

Disposable absorbent articles such as feminine pads, baby diapers, and incontinent garments are typically comprised of several distinct layers, usually at least four or more layers for different product performance functions. Feminine pads, for example, typically comprise: a fluid permeable body side liner (BSL), an intake or surge layer (IL), an absorbency layer and a fluid impermeable outer cover. The BSL is often made of a film or nonwoven material that is in contact with the skin of the wearer to keep the wearer dry and comfortable by keeping the skin separated from the wetness in the layers below. A plurality of apertures may be provided in the BSL to enhance fluid intake. While film and nonwoven materials of a BSL have their advantages and disadvantages, it is an added layer that requires additional materials, treatments, handling, processing and different aspects of costs and time in constructing the article. Furthermore, the BSL is in fact a fluid barrier depending on the type of material, thickness and other factors; hence the need for apertures in some cases. Moreover, substrate materials such as coform, airlaid or spunlace that contain natural fibers e.g. pulp or rayon fibers are generally not used as topsheets on personal care absorbent articles because they tend to feel wet against User skin. These substrates, however, are higher absorbency on a gram per gram basis (g/g) and are generally lower cost than the nonwovens or thermoplastic films currently used as topsheets. It is therefore desirable to use materials like coform, airlaid or spunlace as the top layered sheet on personal care absorbent articles to increase protection and comfort while reducing product cost.

Thus, it is an object of the present invention to provide a disposable absorbent article that provides all of the benefits of traditionally constructed absorbent articles, yet can do so with less materials and layered sheets. It further the object of this invention to provide a polymeric chemistry applied onto traditional intake or surge layers in feminine pads and other personal care articles to provide an enhanced feel yet still have the same function of a typical bodyside liner at a lower cost. In addition to providing the normal protection, the present invention also provides improved softness, feel, texture, visual aesthetics and comfort to the wearer.

### SUMMARY OF THE INVENTION

The present invention relates to a personal care absorbent article comprising at least two substrates each having an internal and external surface, wherein at least one substrate is a fluid permeable bodyside substrate selected from spunbond, meltblown, coform, airlaid, bonded-carded web, spunlace materials and combinations thereof; at least one substrate is an impermeable backsheet; and an absorbent core disposed in between said substrates; wherein at least the external surface of at least one substrate has applied to it a benefit agent selected from an additive composition wherein said additive composition is a polymer dispersion selected from polyolefin dispersions, polyisoprene dispersions, polybutadiene-styrene block copolymer dispersions, latex dispersions, polyvinyl pyrrolidone-styrene copolymer dispersions, polyvinyl alcohol-ethylene copolymer dispersions, and combinations thereof; an enhancement component selected from microparticles, expandable microspheres, fibers, additional polymer dispersions, scents, anti-bacterials, moisturizers, medicaments, soothers and combinations thereof; and combinations thereof.

The personal care absorbent article of the present invention may be a feminine hygiene product, diaper, training pant, adult incontinent product or the like and delivers the benefits of a traditional absorbent article yet provides an article with enhanced softness, comfort and an efficiently advantaged layered sheet system.

### BRIEF DESCRIPTION OF THE DRAWINGS

FIG. 1 is an illustration of a traditional feminine care pad showing the typical four layered sheets: a fluid-permeable topsheet, an intake layer, an absorbent core, and a fluid impermeable outer cover.

FIG. 2 is an illustration of an embodiment of the present invention such as a feminine care pad wherein the pad is absent of the traditional topsheet and only has an intake layer that has been modified with a polymeric benefit agent, an absorbent core and a fluid impermeable outer cover.

FIG. 3 is a schematic view of a creping process according to one embodiment of the present invention.

FIG. 4 is a step-by-step description of a process example, for applying the benefit agent composition to the substrates of the present invention.

### DETAILED DESCRIPTION OF THE INVENTION

While the specification concludes with the claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore; do not include solvents or by-products that may be included in commercially available materials, unless otherwise specified. The term “weight percent” may be denoted as “wt. %” herein. Except where specific examples of actual measured values are presented, numerical values referred to herein should be considered to be qualified by the word “about”.

As used herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

As used herein, the phrase “absorbent article” generally refers to devices which absorb and contain body fluids, and more specifically, refers to devices which are placed against or near the skin to absorb and contain the various fluids discharged from the body and, in particular, viscoelastic fluids. Examples of absorbent articles include, but are not limited to, absorbent articles intended for personal wear, such as diapers; incontinence products; feminine hygiene products, such as feminine napkins, panty liners, tampons, and interlabial pads; other personal garments; and the like.

The term “hydrophilic”, as used herein, refers to surfaces with water contact angles well below 90°.

The term “modified intake layer” refers to nonwoven materials including, but not limited to, spunbond, meltblown, coform, airlaid, bonded-carded web materials, hydroentangled (spunlace) materials, combinations thereof and the like that have been treated with the polymeric composition of the present invention that is described herein. The present invention also presents the modified intake layer as the top substrate of the personal care absorbent article rather than as the second sheet found in traditional absorbent articles.

This invention relates to personal care absorbent articles with an efficient layered sheet system. The personal care article of the present invention comprises at least two substrates; each substrate comprising an internal and external surface. On at least the external surface of either or both substrates of the invention, there is provided a benefit agent for enhancing both protection and softness.

Benefit Agents

Benefit agents are applied to at least the external surface of either or both substrates of the present invention. Particularly, benefit agents are used to produce the modified intake layer of the present invention. Instead of the traditional intake layer of traditional absorbent articles, the present invention has a modified intake layer in that the intake layer is treated with a polymeric composition as described herein. Due to the modified intake layer, the present invention eliminates the need for an additional/traditional material of a topsheet. Benefit agents of the present invention may also be used to treat other substrates/layers of the invention. For example, the benefit agent may be applied to the external surface of the backsheet to add an enhanced softness to the outer cover/backsheet of the invention, such as in one embodiment, the outer material of a diaper. In yet another embodiment, the benefit agent may be applied to the diaper containment flaps and/or front or back waist or leg gasketing (elastic areas) to improve softness. In the same diaper embodiment, the benefit agent may also be applied to the external surface of the diaper backsheet in order to improve softness and enhance the feel of the diaper. FIG. 2 shows one embodiment of the present invention wherein a benefit agent 21 is applied onto the intake layer of a feminine pad to create the modified intake layer 22 of the present invention. Benefit agents may be applied to the fluid permeable bodyside substrate to create the modified intake layer and may do so by adding the benefit agent to the substrate in an amount from about 3%, from about 5%, or from about 10%, to about 20%, to about 15%, or to about 10%, by weight of the substrate. The benefit agent may be an additive composition, an enhancement component, or a combination, as described herein.

I. Additive Composition

In a desired application, the additive composition may be present at a level from about 50 mg/m², or from about 100 mg/m² to about 50,000 mg/m², or to about 10,000 mg/m² or to about 5,000 mg/m². The difference between these suggested ranges is dependent on product end-use. Additive compositions of the present invention may be in the form of a polymer dispersion or a polymer solution as set forth below.

A. Polymer Dispersions

Frothable compositions of water insoluble polymers may be in the form of dispersions. The water insoluble polymer materials that are solids, such as powder, granules, and the like, may be converted into a frothable dispersion by mixing it with water and surfactant(s) under certain processing conditions such as high pressure extrusion at an elevated temperature. The polymer dispersion may then be mixed with air and a foaming agent to convert it into a froth.

Examples of dispersions according to the present invention include, but are not limited to, a polyolefin dispersion such as HYPOD 8510®, commercially available from Dow Chemical, Freeport, Tex., U.S.A.; polyisoprene dispersion, such as KRATON®, commercially available from Kraton Polymers U.S. LLC, Houston, Tex., U.S.A.; polybutadiene-styrene block copolymer dispersion such as Butanol®, commercially available from BASF Corporation, Florham Park, N.J., USA; latex dispersion such as E-PLUS®, commercially available from Wacker, Munich, Germany; polyvinyl pyrrolidone-styrene copolymer dispersion and polyvinyl alcohol-ethylene copolymer dispersion, both are available from Aldrich, Milwaukee, Wis., U.S.A.

The additive composition of the present invention may be commercially available, such as HYPOD 8510® dispersion, from the Dow Chemical Corporation which, consists of water, a polyethylene-octene copolymer, and a copolymer of ethylene and acrylic acid. The polyethylene-octene copolymer may be obtained commercially from the Dow Chemical Corporation under the name AFFINITY® (type 29801) and the copolymer of ethylene and acrylic acid may be obtained commercially from the Dow Chemical Corporation under the name PRIMACOR® (type 59081). PRIMACOR® acts as a surfactant to emulsify and stabilize AFFINITY® dispersion particles. The acrylic acid co-monomer of PRIMACOR® is neutralized by potassium hydroxide to a degree of neutralization of around 80%. Therefore, in comparison, PRIMACOR® is more hydrophilic than is AFFINITY®. In a dispersion, PRIMACOR® acts as a surfactant or a dispersant. Unlike PRIMACOR®, AFFINITY®, as suspended in a dispersion, takes on a form of tiny droplets with a diameter of a few microns. PRIMACOR® molecules surround the AFFINITY® droplets to form a “micelle” structure that stabilizes the droplets. HYPOD 8510® contains about 60% AFFINITY® and 40% PRIMACOR®.

When the dispersion becomes a molten liquid on the dryer's hot surface, AFFINITY® forms a continuous phase and PRIMACOR® a dispersing phase forming islands in the AFFINITY® “ocean.” This phase change is called phase inversion. However, occurrence of this phase inversion depends upon external conditions such as temperature, time, molecular weight of solids, and concentration. Ultimately, phase inversion only occurs when the two polymers (or two phases) have enough relaxation time to allow phase inversion completion. In the present invention, HYPOD 8510® coated film retains a dispersion morphology which indicates there is an incompletion of phase inversion. Benefits of the remaining dispersion morphology include, but are not limited to, a more hydrophilic coating layer due to the exposure of the PRIMACOR® phase; and more improved softness of the coated product due to entrapped air bubbles inside the coated HYPOD 8510® layer which provide extra bulkiness.

The process of the present invention may use a high solid, high viscosity dispersion (from about 10% to about 30%) and may contain a large amount of air bubbles (air volume is at least 10 times more than the dispersion volume). Desirably, the commercially available HYPOD 8510® dispersion (about 42% solids, including both AFFINITY® and PRIMACOR®) has a viscosity around about 500 cps whereas water has a viscosity of around about 1 cps. A dispersion containing about 20% HYPOD 8510® may have a viscosity of around 200 cps, a relatively high viscosity, while a dispersion having less than about 1% HYPOD 8510® may have a viscosity closer to water's viscosity (1 cp). After entrapping a high ratio of air, the viscosity of the frothed HYPOD 8510® dispersion has been increased exponentially compared to the dispersion before being frothed. Another commercially available polymer dispersion may be that which is commercially available from Dow as HYPOD 8102.2®. This polymer dispersion includes only AFFINITY®. It is desirable that the present invention may use a benefit agent wherein the additive composition comprises a dispersion that is from about 10%, or from about 20% or from about 24% to about 27% or to about 30%, or to about 32%, by weight of the benefit agent.

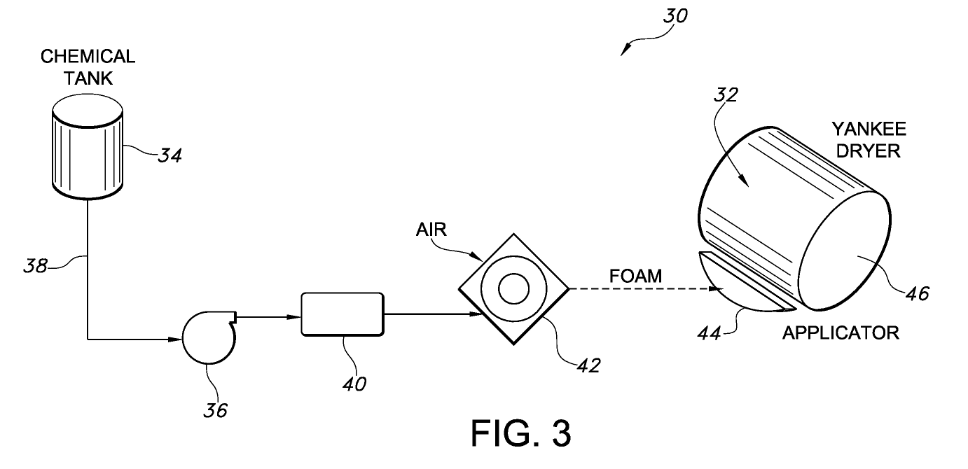

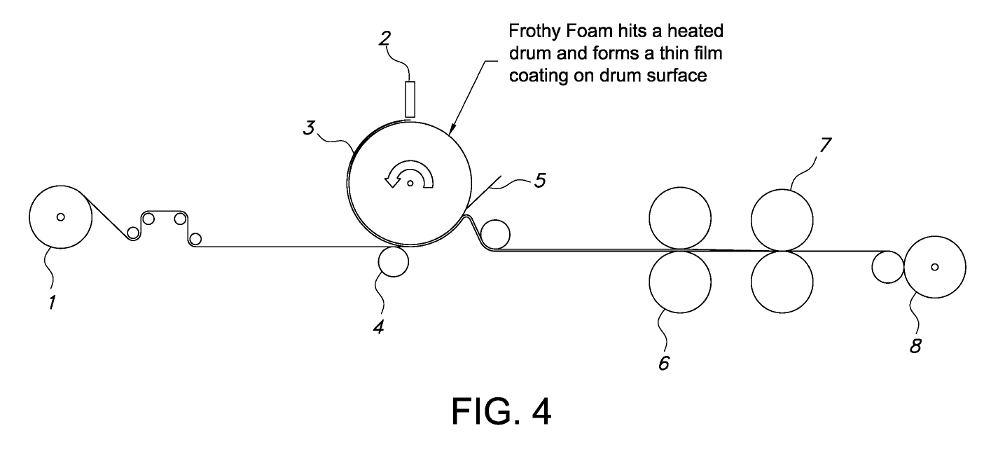

Referring to FIG. 3, when a frothed dispersion is applied onto the non-porous dryer surface 32, a limited amount of water will be quickly evaporated therefrom. It is thought that the dispersion's slow evaporation due to high solids combined with its high viscosity will prevent the AFFINITY®-PRIMACOR® dispersion from completing a phase inversion (wherein the AFFINITY® becomes continuous and the PRIMACOR® becomes a dispersion) and entrapped air from escaping. This results in a unique micro-structured molten film on the hot dryer surface to ultimately transfer the chemistry to the web surface. For examples, the benefit agents of the present invention may be frothed and bonded to the substrate surface through a creping process.

B. Polymer Solutions

Frothable compositions of water soluble polymers may be diluted to form solutions. The water-soluble polymer materials that are solids, such as powder, granules, and the like, may be dissolved into a solution. The polymer solution may then be mixed with air and a foaming agent to convert it into a froth.

Examples of water soluble polymer solutions include both synthetic and natural based water soluble polymers. The synthetic water soluble polymers include, but are not limited to, polyalcohols, polyamines, polyimines, polyamides, polycarboxlic acids, polyoxides, polyglycols, polyethers, polyesters, copolymers and mixtures of the listed above.

The natural based water soluble polymers include, but are not limited to, modified cellulose, such as cellulose ethers and esters, modified starch, chitosan and its salts, carrageenan, agar, gellan gum, guar gum, other modified polysaccharides and proteins, and combinations thereof. In one particular embodiment, the water soluble polymers also include: poly(acrylic acid) and salts thereof, poly(acrylate esters), and poly(acrylic acid) copolymers. Other suitable water soluble polymers include polysaccharides of sufficient chain length to form films such as, but not limited to, pullulan and pectin. For example, the water soluble polymers may contain additional monoethylenically unsaturated monomers that do not bear a pendant acid group, but are copolymerizable with monomers bearing acid groups. Such compounds include, for example, the monoacrylic esters and monomethacrylic esters of polyethylene glycol or polypropylene glycol, the molar masses (Mn) of the polyalkylene glycols being up to about 2,000, for example.

In another particular embodiment, the water soluble polymers may be hydroxypropyl cellulose (HPC) sold by Ashland, Inc. under the brand name of KLUCEL®. The water soluble polymers can be present in the additive composition in any operative amount and will vary based on the chemical component selected as well as on the end properties that are desired. For example, in the exemplary case of KLUCEL®, the biodegradable, water soluble polymers can be present in the additive composition in an amount of about 1% to about 75%, or at least about 1%, at least about 5%, or at least about 10%, or up to about 30%, up to about 50% or up to about 75%, based on the total weight of the additive composition, to provide improved benefits. Other examples of suitable water soluble polymers include methyl cellulose (MC) sold by Ashland, Inc. under the brand name BENECEL®; hydroxyethyl cellulose sold by Ashland, Inc. under the brand name NATROSOL®; and hydroxypropyl starch sold by Chemstar (Minneapolis, Minn., U.S.A.) under the brand name GLUCOSOL 800®. Any of these chemistries, once diluted in water, are disposed onto a hot, non-porous dryer surface to ultimately transfer the chemistry to the web surface. The water soluble polymers in these chemistries include, but are not limited to, polyvinyl alcohol, polyethylene glycol, polyethylene oxide, hydroxypropyl starch, hydroxypropyl cellulose, and combinations thereof.

II. Enhancement Components

The present invention not only provides a personal care absorbent article with an efficient layering system but it also provides improved softness due to the benefit agents and process described herein in addition to an improved hand feel while at the same time employing the same protection offered by traditional personal care absorbent articles. Enhancement components are added to the dispersions of the present invention to provide a cottony/fluffy feel to the substrate instead of the silky/slippery feel that may often be felt with the use of the dispersions alone. While the silky/slippery feel may be desirable for some substrates, the present invention provides other options in order that a variety of textures and aesthetics can be provided. Enhancement components of the present invention include, but are not limited to, micro-particles such as silica gel particles, thermally expandable microspheres such as EXPANCEL®, fibers such as cotton linter flocks, polymer dispersions such as poly(vinylpyrrolidone-styrene), and combinations thereof. When cotton linter flocks or other types of fibers are used, they may be from about 0.1 mm fiber length to about 5 mm fiber length.

In addition to the enhancement components providing a contrasting hand feel, the enhancement components may also provide additional benefits that could not be appreciated with the use of the dispersion alone. Enhancement components of the present invention may also include fragrances, anti-bacterials, moisturizers, soothers, medicaments, surfactants, mucin-modifiers and combinations thereof. Such components will provide an overall substrate that has improved feel, fluid intake performance, and other benefits from the dispersion in combination with benefits that may have not otherwise been provided without the present technology. The present invention may utilize any or a combination of enhancement components to be included within the additive composition of the present invention. For example, enhancement components may be added to a dispersion of the present invention in an amount of from about 0.5%, from about 1% or from about 2% to about 10%, to about 20% or to about 30%, by weight of the dispersion composition.

The enhancement components can be added into the frothed chemistry either before or after the chemistry has been frothed. In a desired application, the enhancement component level is about from about 0.5%, or from about 1%, or about 2% to about 30%, or to about 20%, or to about 10%, based on total dry weight of the benefit agent.

Fluids having viscoelastic properties, such as menses, mucous, blood products, feces and other body fluids for which personal care products are used have a tendency to interfere with the absorption and distribution properties of the personal care products. Thus, a particularly desirable enhancement is a hydrophilic treatment agent added into the frothed chemistry in three various ways: either before or after the chemistry has been frothed or on top of the coating after the frothed coating has been applied to the fluid permeable substrate external surface. Although optional, the hydrophilic treatment agents described herein may exert various combinations of effects on viscosity, elasticity, and fouling of a viscous fluid such as menses or runny BM (feces) depending on the concentration and deposition at which they are added into the dispersion or applied to the substrate. The hydrophilic treatment agent of the present invention may be selected from the group consisting of polyethylene glycol laurates, polyethylene glycol lauryl ethers, and combinations thereof. Advantageously, the polyethylene glycol laurates and polyethylene glycol lauryl ethers are capable of reducing both the viscosity and elasticity of viscoelastic fluid. Examples of suitable polyethylene glycol laurates include, but are not limited to, polyethylene glycol 400 monolaurate, polyethylene glycol 600 monolaurate, polyethylene glycol 1000 monolaurate, polyethylene glycol 4000 monolaurate, polyethylene glycol 600 dilaurate, and combinations thereof. Examples of suitable polyethylene glycol lauryl ethers include, but are not limited to, polyethylene glycol 600 lauryl ether. Notably, the polyethylene glycol lauryl ether and/or polyethylene glycol laurate serves not only as a hydrophilic treatment agent, but is further capable of reducing the fouling properties of viscoelastic fluid. Such hydrophilic treatment agents include, but are not limited to, polyethylene glycol (PEG) 600 lauryl ether and related compounds, polyethylene glycol (PEG) 600 monolaurate and related compounds, and combinations thereof. A particular beneficial PEG 600 lauryl ether is sold under the trade name LUTENSOL A 65 N by BASF Corporation.

In addition to the PEG laurates and PEG lauryl ethers, other polyethylene glycol derivatives may be viscoelastic agents (i.e., are capable of reducing the viscosity and elasticity of viscoelastic fluids) and may be used as hydrophilic treatment agents for the personal care products described herein. As used herein, the term “polyethylene glycol derivative” includes any compound comprising a polyethylene glycol moiety. Examples of other suitable PEG derivatives include, but are not limited to, PEG monostearates such as PEG 200 monostearates and PEG 4000 monostearate; PEG dioleates such as PEG 600 dioleate and PEG 1540 dioleate; PEG monooleates such as PEG 600 monooleate and PEG 1540 monooleate; PEG monoisostearates such as PEG 200 monoisostearate; and PEG 16 octyl phenyl. Particular polyethylene glycol derivatives for use as hydrophilic treatment agents are those that improve intake time of viscoelastic fluids as well as reduce viscosity and elasticity. Examples of such include, but are not limited to, PEG derivatives include PEG 1540 dioleate, PEG 600 monooleate, PEG 1540 monooleate, and PEG 16 octyl phenyl. These PEG derivatives may be used alone or in combination with PEG 600 monolaurate, PEG 600 lauryl ether, and/or other viscoelastic agents as a hydrophilic treatment agent.

In certain embodiments, the hydrophilic treatment agents described herein, such as polyethylene glycol 600 lauryl ether and/or the polyethylene glycol 600 monolaurate, may be used in combination with each other or in combination with other viscoelastant agents. Examples of additional viscoelastant agents that may be used in combination with the hydrophilic treatment agents include, but are not limited to, sodium citrate, dextran, cysteine, Glucopon 220UP (available as a 60% (by weight) solution of alkyl polyglycoside in water from Henkel Corporation), Glucopon 425, Glucopon 600, Glucopon 625. Other suitable viscoelastant agents are described in U.S. Pat. No. 6,060,636. Surprisingly, it has been discovered that certain viscoelastant agents that actually increase the fouling effect of viscoelastic fluids when used alone, will in fact improve fouling effects when used in combination with PEG 600 lauryl ether and/or PEG 600 monolaurate. For example, in one embodiment, sodium citrate may be used in combination with PEG 600 monolaurate as a hydrophilic treatment agent. When two or more hydrophilic treatment agents are used in combination, the proportion of each hydrophilic treatment agent applied to the personal care product is preferably in a ratio of from about 1:2 to about 2:1, and more preferably is about 1:1.

When the benefit agent comprises enhancement components in combination with the additive compositions of the present invention, they allow for enhanced softness and absorbency without compromising strength. The benefit agent of the present invention may be made according to the following table wherein the various components are included at a particular percentage, by weight of the benefit agent. For example, a benefit agent “A”, as shown in the table, may comprise from about 24% of an additive composition such as a polymer dispersion like HYPOD 8510®, 6% of an enhancement component like silica gels such as Syloid 244® and 70% water. Other benefit agents of the present invention may be included in the present invention as exemplified in Table 1.

TABLE 1
Benefit Agent Compositions

| Benefit | | Enhancement | | |
|---|---|---|---|---|
| Agent | Additive Composition | Components | Component 3 | Surfactants |
| A | 24% HYPOD 8510 | 6% Syloid 244 FP | 70% Water | N/A |
| | | Silica Gel | | |
| B | 24% HYPOD 8510 | 6% Syloid 244 FP | 65% water | 5% Lutensol |
| | | Silica Gel | | A65N, non- |
| | | | | ionic surfactant |
| C | 30% HYPOD 8510 | 2% KLUCEL | 63% water | 5% Lutensol |
| | | Hydroxypropylcellulose | | A65N, non- |
| | | (HPC) from Hercules, | | ionic surfactant |
| | | Inc. | | |
| D | 30% HYPOD 8510 | | 65% water | 5% Lutensol |
| | | | | A65N, non- |
| | | | | ionic surfactant |
| E | 30% HYPOD 8102.2 | 2% KLUCEL | 63% water | 5% Lutensol |
| | | Hydroxypropylcellulose | | A65N, non- |
| | | (HPC) from Hercules, | | ionic surfactant |
| | | Inc. | | |
| F | 30% HYPOD 8102.2 | | 65% water | 5% Lutensol |
| | | | | A65N, non- |
| | | | | ionic surfactant |

III. Processing Aids

Processing aids of the present invention include chemicals that may help in the process of forming the treated substrate of the present invention. The processing aids may slightly appear or may dissipate in the final, treated substrate. While they are included to solely aid in the process of producing the treated substrates, they may also impart slight benefits to the substrate that are desired of the present invention. For the purposes of this application, “processing aids” are those used in the process of frothing or applying the benefit agents to the substrate and are not used in the process of making the precursor substrate. An example of a processing aid is Unifroth 0154, anionic surfactant available from Unichem, Inc of Haw River, N.C.

A. Foaming Agents

Most commercial foaming agents are suitable for creating the froth of the present invention. Suitable foaming agents include, but are not limited to, either low molecular or polymeric materials in liquid form. The foaming agents can be anionic, cationic or nonionic. These foaming agents can be divided into four groups depending on function:

1. Air Entrapment Agent—used to enhance a liquid's (dispersion, solution, or a mixture, etc.) capability to entrap air which can be measured by determining a “blow ratio.” An exemplary list of foaming agents include but is not limited to potassium laurate, sodium lauryl sulfate, ammonium lauryl sulfate, ammonium stearate, potassium oleate, disodium octadecyl sulfosuccinimate, hydroxypropyl cellulose, etc.

2. Stabilization Agent—used to enhance stability of froth's air bubbles against time and temperature; examples include, but are not limited to, sodium lauryl sulfate, ammonium stearate, hydroxypropyl cellulose, etc.

3. Wetting Agent—used to enhance the wettability of a film-coated dried surface. Examples include, but are not limited to, sodium lauryl sulfate, potassium laurate, disodium octadecyl sulfosuccinimate, etc.

4. Gelling Agent—used to stabilize air bubbles in the froth by causing the additive composition to take the form of a gel which serves to reinforce cell walls. Examples include, but are not limited to, hydroxypropyl cellulose, hydroxyethyl cellulose, carboxymethyl cellulose and other modified cellulose ethers.

Some foaming agents can deliver more than one of the functions listed above. Therefore, it is not necessary to use all four foaming agents in a frothable additive composition. Selection of the foaming agents is dependent upon the chemistry of the additive composition. For example, when the additive composition comprises an anionic component, such as HYPOD 8510®, suitable foaming agents have to be selected from either anionic or non-ionic groups. If a cationic foaming agent is used to enhance frothability of an anionic additive composition, the cationic components in the foaming agent will form ionic bonds with the anionic components in the additive composition and cause both cationic foaming agent and anionic additive composition to become water insoluble due to formation of the bonds. On the other hand, if an additive composition comprises cationic components, anionic foaming agents are not suitable to use.

B. Creping Aids

Creping Aids are chemistries that are added to the benefit agents of the present invention to optimize the adhesion and release properties of the substrate to the dryer surface. These fall broadly into the following groupings:
1. Adhesion Aid—used to increase adhesion of the substrate to the dryer surface.

Examples include, but are not limited to, polyvinyl alcohol, polyacrylate, hydroxypropy starch, carboxymethy cellulose, kymene, polyvinyl amine, copolymers or mixtures thereof.
2. Release Aid—used to decrease adhesion (enhance release) of the substrate to (from) the dryer surface. Examples include, but are not limited to, polyethylene glycol, polypropylene glycol, polyethylene oxide, polypropylene oxide, polyolefin, fluorinated polyolefin, copolymer and blends comprising the above.
3. Curing Aid—used to hasten or retard curing of the creping package such as a plasticizer or toughener

Froth Generating Process

In general, preparing frothed chemicals utilizes a system that pumps both liquid and air into a mixer. The mixer blends the air into the liquid to produce a froth which inherently includes a plurality of small air bubbles. The froth exits the mixer and flows to an applicator.

One parameter to define the quality of frothed chemistry is the blow ratio, which is defined by ratio of volume of small air bubbles entrapped by dispersion chemical to the volume of the dispersion before mixing. For example, at a blow ratio of 10:1, a dispersion flow rate of 1 liter/minute will be able to entrap 10 liters/minute of air into its liquid and produce a total froth flow rate of 11 liters per minute.

To achieve a high blow ratio, both the mechanical mixing and the frothing capability of the additive composition are determining factors. If a chemical can only hold or entrap air volume up to a blow ratio of 5, no matter how powerful a froth unit is, it won't be able to produce a stable froth having a blow ratio of 10. Any extra air beyond the blow ratio of 5 will release out of the froth system once the mechanical force is removed. In other words, any entrapped air higher than the dispersion's air containment capability will become instable. Most of such instable air bubbles will escape from the froth (debubbling) immediately after mechanical agitation is stopped.

Referring to FIG. 3, shown schematically, is a system 30 that can generate the frothed chemistry according to the present invention. To begin, frothable chemicals (e.g. HYPOD 8510®, KRATON®, and the like) are placed in a chemical tank 34. The chemical tank 34 is connected to a pump 36. It may be desirable to modify piping 38 between the chemical tank 34 and pump 36 so that one may transmit the frothable chemicals to two different sizes of pumps. Desirably the chemical tank 34 is situated at an elevated level above the pump 36 in order to keep the pump primed.

One optional small secondary pump (not shown) may be used for running the frothing process at slow speeds relative to the pump 36. The larger primary pump 36 is capable of producing flow rates up to 25 liters/minute liquid flow-rate for high application speeds and/or high amounts of additive composition. The smaller, secondary pump (not shown) is capable of liquid flow rates up to about 500 cc/min. for low application speeds and/or low additive composition.

A flow meter 40 is situated between the pump(s) 36 and a foam mixer 42. Liquid flow rates are calculated from desired additive composition, chemical solids, line-speed and applicator width. The flow rate may range from about 5:1 to about 50:1. When using the small secondary pump, its flow rate ranges from about 10 cc/min to about 500 cc/min. When using the large pump 36, its flow rate ranges from about 0.5 liter/min to about 25 liter/min. A 20 liter/min air flow meter (not shown) is selected when using the small secondary pump (not shown). There is a 200 liter/min air flow meter 40 to use when running the larger primary pump 36.

In one aspect, the foam mixer 42 is used to blend air into the liquid mixture of frothable chemicals to create small air bubbles in the froth. Air is metered into the system 30 using certain liquid flow rates and blow ratios as discussed above. Desirably, the foam mixer 42 having a size of 25.4 cm (10 inches) may be used to generate froth. One possible foam mixer 42 is a CFS-10 inch Foam Generator from Gaston Systems, Inc. of Stanley, N.C., U.S.A.

Desirably, the rotational speed of the foam mixer 42 is limited to about 600 rpm. The rpm speed for the mixer in this process is dependent upon the additive composition's ability to foam (i.e., its capability of entrapping air to form stable bubbles). If the additive composition foams easily, a lower rpm is generally required. If the additive composition does not foam easily, a higher rpm is generally required. The higher mixer speed helps to speed up the foam equilibrium or optimal blow ratio. A normal rpm for the mixer is about 20%-60% of the maximum rpm speed. The type of and/or amount of foam agent in addition to the additive composition also has an effect on the mixer speed requirement.

The froth is checked for bubble uniformity, stability and flow pattern. If bubble uniformity, stability and flow pattern are not to desired standards, adjustments may be made to flow rates, mixing speeds, blow ratio, and/or chemical compositions of the solutions/dispersions before directing the froth to the applicator 44.

In one aspect of the invention, HYPOD 8510®, or other chemistries to be frothed and used for creping are blended and added to the chemical tank 12. Dilute solutions of HYPOD 8510® (<10% total solids) and other hard-to-froth chemistries generally require something added to the formulation to increase viscosity and foamability. For example, hydroxypropyl cellulose or other foaming agents or surfactants, can be used to produce a stable froth for uniform application onto the heated and non-permeable surface of a rotating drum of a dryer surface. The enhancement components, such as silica gel particles or cotton linter flocks, can be added into the additive composition in various ways, including, but not limited to: added into the additive composition before the additive composition is pumped into a frothing machine; introduced into the frothed additive composition after the additive composition is coming out of the frothing machine but before the frothed additive composition is applied onto the dryer's surface; or applied to the dryer before the substrate contacts the additive composition. When the enhancement components are introduced into the additive composition, it is necessary to constantly agitate the mixture before adding it into the frothing machine in order to prevent the solid enhancement component from being settled down at the bottom of the container. When the enhancement components are introduced into the frothed additive composition, a suitable device, which ensures a uniform mixing of the enhancement components and the frothed additive composition, is needed.

Substrates

The present invention relates to a personal care absorbent article comprising at least two substrates. Each of the two substrates has an internal and external surface. At least one substrate is a fluid permeable bodyside substrate. Specifically, the permeable bodyside substrate of the present invention is a single modified intake layer selected from spunbond, meltblown, coform, airlaid, bonded-carded web, spunlace materials and combinations thereof. The other substrate is an impermeable backsheet. At least one substrate has a benefit agent applied on at least its external surface wherein the benefit agent is selected from an additive composition, an enhancement component and combinations thereof. The benefit agent may be applied onto the substrate at a level from about 50 mg/m2 to about 50,000 mg/m2, or from about 50 mg/m2 to about 10,000 mg/m2 or from about 100 mg/m2 to about 5,000 mg/m2. The difference between these suggested ranges is dependent on product end-use. The present invention also comprises an absorbent core positioned between the substrates.

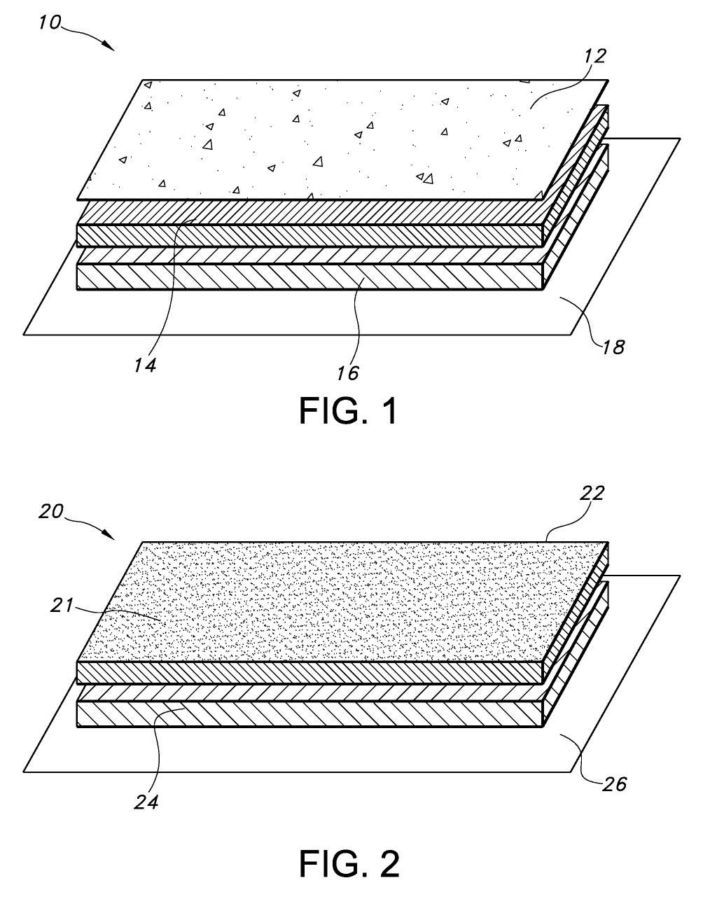

The personal care absorbent article of the present invention may be a product selected from a variety of personal care absorbent articles including, but not limited to feminine pads, adult incontinence articles, diapers, training pants and the like that eliminates the need for a traditional nonwoven topsheet due to the aforementioned benefit agent that not only enhances the feel of the overall article but provides efficiency in the layering system of the overall article. The present invention therefore relies on a single modified intake layer material to perform the dual function of wetness separation and fluid intake management currently performed by two separate material layers, namely a nonwoven or film body-side liner 12 against the user skin and an intake Layer 14 as shown in FIG. 1 in order to provide the reduced cost, efficient personal care absorbent article as shown, by example in FIG. 2.

Traditional absorbent articles, such as the feminine care absorbent product 10, illustrated in FIG. 1, for example, may include a liquid permeable topsheet 12, a substantially liquid impermeable backsheet 18 joined to the topsheet 12, and an absorbent core 16 positioned and held between the topsheet and the backsheet. The topsheet is operatively permeable to the liquids that are intended to be held or stored by the absorbent article for example through apertures extending from the external surface through the internal surfaces; and the backsheet may be substantially impermeable or otherwise operatively impermeable to the intended liquids. In addition to the absorbent core 16, the traditional feminine care absorbent product may also include an additional layered sheet known as the intake layer 14. The intake layer 14 may also be traditionally referred to as the liquid intake layer, liquid wicking layers, liquid distribution layers, transfer layers, barrier layers, and the like, as well as combinations thereof. Disposable absorbent articles and the components thereof can operate to provide a body-facing surface (external surface of the traditional topsheet or the external surface of the modified intake layer as described herein) and a garment-facing surface (external surface of the backsheet). As used herein, the “body-facing” or “bodyside” surface refers to the surface that is disposed toward or placed adjacent to the body of the wearer during ordinary use. For the present invention, the bodyside surface is the external surface of the modified intake layer. The “garment-side surface” refers to the external surface of the outer cover or backsheet where the surface is disposed away from the wearer's body and adjacent to the garment of the wearer during ordinary use. Absorbent articles are also described in more detail in U.S. Pat. No. 7,632,258 to Misek et al.

In summary, typical absorbent articles 10 comprise at least three layered sheets consisting of a topsheet 12, absorbent core 16 and a backsheet 18. Most absorbent articles of today also consist of at least four layered sheets since they further comprise a liquid intake layer 14. The present invention provides efficient manufacturing without compromising the performance of an absorbent article as it eliminates the need for a traditional fluid permeable topsheet 12.

Although the present disclosure is discussed primarily in combination with feminine hygiene products such as feminine napkins, panty liners, and interlabial pads, it will be readily apparent to one skilled in the art based on the disclosure that the products and methods described herein can also be used in combination with numerous other absorbent articles designed to absorb fluids other than menses such as, but not limited to, diapers, incontinence articles and training pants for runny BM, urine and the like.

Referring to FIG. 2, an absorbent article of the present disclosure is representatively illustrated in the form of a feminine/incontinence pad and is indicated in its entirety by the reference numeral 20. The exemplary feminine/incontinence pad includes a backsheet having a garment-side, external surface (otherwise referred to as a baffle or backsheet) 26, an absorbent core 24 and a modified intake layer 22 having a bodyside, external surface.

Modified Intake Layer

As shown in FIG. 2, the traditional fluid permeable topsheet is absent in the present invention. The disposable absorbent articles of the present invention are particularly adapted to receive fluids having viscoelastic properties, such as menses, mucous, blood products, and feces, among others to reduce stain area, reduce rewet, improve fluid intake, distribution, absorption properties and decrease leakage through a single, intake layer that has been treated with the benefit agent 21 composition of the present invention. Because the external surface of the intake layer has been treated with the benefit agent 21, the intake layer of the present invention is most suitably referred to as a modified intake layer 22. The present invention also provides for at least an absorbent core 24 and a backsheet 26, as shown in the embodiment of FIG. 2.

The basis weight of the modified intake layer of the present invention is determined in grams per square meter (gsm) and may be in the range of from about 10 gsm, from about 25 gsm, or from about 25 gsm to about 100 gsm, to about 150 gsm, or to about 200 gsm. For example, the modified intake layer of the present invention may range from about 14 gsm to about 200 gsm.

The present invention still provides advantages in protecting against stains, re-wetting and minimizing particulate debris accumulation from body waste on the body-facing surface and other unpleasantries. For example, the modified intake layer 22 may have a saline, menses or other bodily fluid waste absorbent capacity (grams of liquid/grams of modified intake layer (g/g)) greater than about 1 g/g, or greater than about 5 g/g, or greater than about 10 g/g depending on thickness, density and other material structural factors such as permeability. The modified intake layer absorbent capacity can be determined using a method such as the Modified Retention Capacity (mCRC) Test described below:

Modified Centrifuge Retention Capacity (mCRC) Test

This test can be used to determine absorbent capacity of the modified intake layer of the present invention while in limited liquid condition. The resultant retention capacity is stated as grams of liquid retained per gram weight of the sample (g/g). In this method, 2.0 to 10±0.050 g of dry modified intake layer is inserted into a tea bag. A heat-sealable tea bag material, such as that available from Dexter Corporation (having a place of business in Windsor Locks, Conn., U.S.A.) as model designation 1234T heat sealable filter paper works well for most applications. The bag is formed by folding a 5-inch by 3-inch (12.7-cm×7.6-cm) sample of the bag material in half and heat-sealing two of the open edges to form a 2.5-inch by 3-inch (6.4-cm×7.6-cm) rectangular pouch. The heat seals are about 0.25 inches (0.6 cm) inside the edge of the material.

After the sample is placed in the pouch, the remaining open edge of the pouch is also heat-sealed. Empty bags can also be made to serve as controls. 10 ml of saline solution (i.e., 0.9 wt % aqueous sodium chloride) is placed into a container, sufficiently large to permit the teabag to lay flat, yet small enough to prevent the saline from spreading over an excessively large area. The container for the saline should have a bottom cross-sectional area between 8 in2-15 in2 (52 cm2-97 cm2). An appropriate container is a 100 mm diameter Petri dish, catalog number 25384-056 available from VWR International (having a place of business located in West Chester, Pa., U.S.A.). The teabag is placed in the saline solution for a fixed period of time, such as 5 minutes, making sure that the bags are held down until they are completely wetted. Following the fixed period of immersion in saline, the teabag is centrifuged for 3 minutes at 290G-force with a variance from about 286 to about 292G-force). G-force is defined as a unit of inertial force on a body that is subjected to rapid acceleration or gravity, equal to 32 ft/sec/sec at sea level.

The absorbed quantity of saline solution is determined by measuring the weight of the teabag. The amount of solution retained by the modified intake layer sample, taking into account the solution retained by the bag itself, is the absorbent capacity of the modified intake layer sample at the fixed immersion time, expressed as grams of fluid per gram of sample. More particularly, the absorbent capacity is determined by the following equation:

$$\frac{\begin{matrix}[ sample and bag wt . after centrifuge ] - \\ [ empty bag wt . after centrifuge ] - \\ [ dry sample wt . ]\end{matrix}}{[ dry sample wt . ]}$$

In order to fully characterize the absorbent capacity under limited liquid conditions, multiple samples of modified intake layer material need to be prepared as described above and placed into multiple teabags. Each teabag must be immersed in its own 10 ml of saline solution. The time an individual sample is immersed in the saline solution should range from 5 minutes to 20, at 5 minute intervals. Each immersion time can be done with only one replicate and the absorbent capacity calculated for each immersion time and averaged.

The benefit agent 21 is applied to the external surface of the modified intake layer 22 or the external surface of the backsheet 26. Although the present invention prefers that the benefit agent is creped onto the modified intake layer 22, it is to be noted that the benefit agent can also be applied onto the substrate in a variety of other ways including, but not limited to, sprayed, printed, slot-coated or kiss rolling order to arrive at the modified intake layer 22 of the present invention. An example of applying the benefit agents onto the substrates is described in co-pending U.S. application Ser. No. 13/330,440 filed Dec. 19, 2011 and U.S. application Ser. No. 12/979,852 filed Dec. 28, 2010, both to Qin et al.

Unlike other articles that provide coating and the like onto a substrate for enhanced benefits, the present invention provides uniqueness in that it not only provides an efficiently layered article but it also provides an enhanced softness. Most notably, the compositions of the benefit agents described herein provide such softness without the composition transferring from the substrate onto the user's skin. The unique compositions of the benefit agents may be bonded onto the external substrate surface via the process described herein and in co-pending U.S. application Ser. No. 13/330,440 filed Dec. 19, 2011 and U.S. application Ser. No. 12/979,852 filed Dec. 28, 2010, both to Qin et al. Thus, unlike lotions, medicaments, and other compositions that have been coated onto a substrate and then transferred to the user's skin to give a softer feel, healing properties, or the like, the present invention provides such advantages without the necessity of compositions leaving the substrate and being deposited on the wearer's body, garments, etc. . . .

The modified intake layer 22 can be in a variety of shapes and configurations known in the art, such as rectangular, hourglass shaped, I-shaped, and the like; and can be provided with a plurality of apertures of different shapes, sizes and hole density (holes/in²) extending from the external surface through the internal surface to facilitate fluid passage through the modified intake layer 22 to the absorbent core and other layers intermediate between the modified intake layer 22 and backsheet 26. The modified intake layer 22 further has opposed lateral edges (not shown) and opposed longitudinal ends (not shown). The lateral edges and longitudinal ends together make up the perimeter of the absorbent assembly. The modified intake layer 22 is designed to take in body exudates, including menstrual fluid, blood, urine, and other body fluids. The modified intake layer 22 may be comprised of a variety of materials. Suitable materials for the modified intake layer 22 include, spunbond, meltblown, coform, airlaid, bonded-carded web, spunlace materials and combinations thereof. A desired modified intake layer 22 material may be selected from an airlaid, spunlace or coform material that has been treated with the benefit agent described herein.

The modified intake layer of the present invention may further comprise apertures to help keep fluids from sitting atop the surface which can leave an unpleasant and/or unclean feeling from stains, accumulated debris or wetness on the surface. The modified intake layer may comprise said apertures to better receive fluids having viscoelastic properties, such as menses, mucous, blood products, and feces, among others to reduce stain area, reduce rewet, improve fluid intake, distribution, absorption properties and decrease leakage. The apertures may vary in shape, size and overall distribution in the number and placement of apertures within the modified intake layer. Aperture diameters may range from about 0.1 mm to about 5 mm, from about 0.4 mm to about 1.5 mm or from about 0.5 to about 1.0 mm. The aperture density may also range from about 7.8×10³ holes/m² to about 1.9×10⁶ holes/m², from about 4.7×10⁴ holes/m² to about 9.3×10⁵ holes/m² or from about 1.6×10⁵ holes/m² to about 3.1×10⁵ holes/m². The apertures may extend through the modified intake layer from the external surface of the bodyside surface through and to the internal surface of the modified intake layer. Furthermore, the external surface of the modified intake layer 22 may be provided with embossing designs of different shapes, sizes and patterns to enhance personal care article visual aesthetics and or channel fluids to said apertures for absorption into the absorbent core 24 below the modified intake layer 22.

Absorbent Core

The absorbent core 24 of the present invention includes a superabsorbent material, which increases the ability of the absorbent article to absorb a large amount of fluid in relation to its own weight. Generally stated, the superabsorbent material can be a water-swellable, generally water-insoluble, hydrogel-forming polymeric absorbent material, which is capable of absorbing at least about 15, suitably about 30, and possibly about 60 times or more its weight in physiological saline (e.g. saline with 0.9 wt % NaCl). The superabsorbent materials can be inserted as particles or in sheet form. The superabsorbent material can be biodegradable or bipolar. The superabsorbent material can be formed from organic hydrogel-forming polymeric material, which can include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers can be lightly crosslinked to render the material substantially water insoluble. Crosslinking can, for example, be by irradiation or covalent, ionic, Van der Waals, or hydrogen bonding. Hydroxyfunctional polymers have been found to be good superabsorbents for sanitary napkins Such superabsorbents are commercially available from Evonik Stockhausen, LLC, Greensboro, N.C., U.S.A.; BASF Corporation, Florham Park, N.J., U.S.A.; and Nippon Shokubai, Chattanooga, Tenn., U.S.A., among others, and are a partially neutralized salt of cross-linked copolymer of polyacrylic acid and polyvinyl alcohol having an absorbency under load value above 25 grams of absorbed liquid per gram of absorbent material (g/g). Other types of superabsorbent materials known to those skilled in the art can also be used.

Backsheet

The backsheet can be constructed of any operative material, and can have selected levels of liquid-permeability or liquid-impermeability, as desired. For example, the backsheet can be configured to provide an operatively liquid-impermeable baffle structure. The backsheet, for example, may include a polymeric film, a woven fabric, a nonwoven fabric or the like, as well as combinations or composites thereof. For example, backsheet can include a polymer film laminated to a woven or nonwoven fabric. In a particular feature, the polymer film can be composed of polyethylene, polypropylene, polyester or the like, as well as combinations thereof. Additionally, the polymer film can be micro-embossed, have a printed design, have a printed message to the consumer, and/or can be at least partially colored. Suitably, the backsheet can operatively permit a sufficient passage of air and moisture vapor out of the article while blocking the passage of bodily liquids. An example of a material suitable for the backsheet can include a breathable, microporous film, such as those described in, for example, U.S. Pat. No. 6,045,900 to McCormack et al. Bicomponent films or other multi-component films can also be used, as well as woven and/or nonwoven fabrics that have been treated to render them operatively liquid-impermeable. Another suitable backsheet material can include closed-cell polyolefin foam. Backsheets of the present invention may have applied onto the external surface of the garment-side facing surface the benefit agents of the present invention. Such treatment may provide the user with enhanced protection and softer feel when compared to untreated surfaces.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Test Methods

### (1) In-Hand Ranking Test for Tactile Properties (IHR Test):

The In-Hand Ranking Test (IHR) is a basic assessment of in-hand feel of fibrous webs and assesses attributes such as softness. This test is useful in obtaining a quick read as to whether a process change is humanly detectable and/or affects the softness perception, as compared to a control. The difference of the IHR softness data between a treated web and a control web reflects the degree of softness improvement.

A panel of testers was trained to provide assessments more accurately than an average untrained consumer might provide. Rank data generated for each sample code by the panel were analyzed using a proportional hazards regression model. This model computationally assumes that the panelist proceeds through the ranking procedure from most of the attribute being assessed to least of the attribute. The softness test results are presented as log odds values. The log odds are the natural logarithm of the risk ratios that are estimated for each code from the proportional hazards regression model. Larger log odds indicate the attribute of interest is perceived with greater intensity.

Because the IHR results are expressed in log odds, the difference in improved softness is actually much more significant than the data indicates. For example, when the difference of IHR data is 1, it actually represents 10 times (10¹=10) improvement in overall softness, or 1,000% improvement over its control. In another example, if the difference is 0.2, it represents 1.58 times (10^{0.2}=1.58) or a 58% improvement.

The data from the IHR can also be presented in rank format. The data can generally be used to make relative comparisons within tests as a product's ranking is dependent upon the products with which it is ranked. Across-test comparisons can be made when at least one product is tested in both tests.

(2) Modified Centrifuge Retention Capacity (mCRC) Test

The modified centrifuge retention capacity (mCRC) Test can be used to determine absorbent capacity of the modified intake layer of the present invention while in limited liquid condition. As described herein, the resultant retention capacity is stated as grams of liquid retained per gram weight of the sample (g/g). More particularly, the absorbent capacity is determined by the following equation:

$$\frac{\begin{matrix}[ sample and bag wt . after centrifuge ] - \\ [ empty bag wt . after centrifuge ] - \\ [ dry sample wt . ]\end{matrix}}{[ dry sample wt . ]}$$

In order to fully characterize the absorbent capacity of the MIL under limited liquid conditions, multiple samples of the modified intake layer (MIL) need to be prepared as described above and placed into multiple teabags. Each teabag must be immersed in its own 10 ml of saline solution. The time an individual sample is immersed in the saline solution should range from 5 minutes to 20 minutes at 5 minute intervals. Each immersion time can be done with only one replicate and the absorbent capacity calculated for each immersion time.

### Example 1

A sensory panel of 12 female panelists ages 21-65 evaluated softness of selected nonwoven substrates (Table 2) coated with the benefit agent “A” (Table 1) of the present invention comprising about 70% water, 24% HYPOD 8510 polyolefin dispersion available from DOW Chemical and 6% Silica Gel available from Sigma Aldrich or Grace Davidson compared to non-coated substrates (Table 2). The panelists were instructed to set the samples on mats in the specified order with the blinding codes facing up. (Note: “Blinding Codes” are, for example, three digit numbers such as ‘365’ used to “blind” the sample so that the panelists are only able to identify the sample by a number rather than by composition. The panelists rank four samples at a time and evaluate the samples in a particular order. For example, four substrates may be evaluated from left to right by ‘365’, ‘428’, ‘209’ and ‘731’. A panelist evaluates each sample and then arranges them according to the attribute she has been asked to evaluate.)

Using a circular motion, they were instructed to stroke each sample with the pads of the fingers on their dominant hands (index, middle, and ringer finger). They were then asked to rank the samples from most to least for feeling soft. Results of Table 3 indicate that codes 35A, 16, 3 and 13, all coated with approximately 20% add-on of the benefit agent based on the substrate weight (grams of agent to gram of substrate) were ranked higher for softness over the traditional non-coated topsheets substrates (code 11—film; and code22—spunbond nonwoven) used in two commercial feminine menstrual hygiene pads.

Code 35A was a 24 gsm (grams per square meter) Through-air Bonded Carded Web TABCW) material coated with the benefit agent composition of the present invention and provided with apertures; code 16 was a non-apertured 40 gsm coform comprising 40% Vistamaxx 2330 polymer meltblown fibers, 60% CF405 pulp fibers from Weyerhaeuser, and 3% TECHSURF 15560 surfactant; code 13 Spunlace and code 3 was a proprietary nonwoven laminate.

TABLE 2
Substrates
Substrate Material Examples as indicated in Table 3

1: FRF pre-apertured proprietary nonwoven laminate non-coated with
Agent (A), provided with a plurality of additional apertures
2: FRF proprietary nonwoven, Freudenberg, Germany, agent (A) coated,
additional apertures
3: FRF, pre-apertured proprietary nonwoven laminate from Freudenberg,
agent coated, no additional apertures
5A: FIB, proprietary nonwoven laminate from Fiberweb, agent A coated,
no apertures
8A: 24 gsm Kimberly-Clark TABCW, agent A coated, large apertures
through Hypod side
9A: 24 gsm Kimberly-Clark TABCW, agent A, large apertures through
nonwoven side
9B: 24 gsm Kimberly-Clark TABCW, agent A coated, small apertures
through nonwoven side
13: spunlace nonwoven, agent (A) coated, no apertures
14: Spunbond-meltblown-spunbond laminate from Kimberly-Clark,
agent A coated, apertures
16: 40 gsm Coform, agent (A), no apertures
19: 20 gsm spunbond nonwoven, agent A, apertures
20: 20 gsm spunbond from Fiberweb, France, agent A coated, apertures
22: 20 gsm spunbond from Fiberweb, France, non-coated, no apertures
25: Traditional apertured film used as topsheet in commercial feminine
pads different aperture sizes than code 11
26: 24 gsm TABCW, agent (A) coated and provided with wave aperture
patterns
28: 24 gsm TABCW from Kimberly-Clark, agent (A) coated, no apertures
35: 25 gsm TABCW, agent (A) coated, apertures
(stiffer material due to higher nip pressure
and dryer temperatures to increase coating adhesion to substrate)
35A: 25 gsm TABCW, agent (A) coated, apertures (softer material)

*TABLE 3
Softness of Substrates Ranking

| | Overall | Log | Standard | 95% |
|---|---|---|---|---|
| Code | Probability | Odds | Error | Grouping |
| 35A | 32.5% | 6.2175 | 0.5124 | A |
| 16 | 14.0% | 5.372 | 0.4538 | B |
| 3 | 12.3% | 5.2432 | 0.4706 | B |
| 13 | 11.4% | 5.169 | 0.4549 | B |
| HIGH | 9.0% | 4.9355 | 0.4564 | B |
| 28 | 8.8% | 4.9127 | 0.4373 | B |
| 26 | 2.2% | 3.5041 | 0.4311 | C |
| 1 | 2.0% | 3.432 | 0.4233 | C |
| 8A | 1.9% | 3.3996 | 0.4298 | C |
| 9B | 1.5% | 3.1715 | 0.4162 | CD |
| 19 | 0.9% | 2.636 | 0.4123 | DE |
| 2 | 0.8% | 2.5345 | 0.4087 | DEF |
| 14 | 0.7% | 2.34 | 0.3964 | EFG |
| 20 | 0.4% | 1.8344 | 0.4053 | FGH |
| 35 | 0.4% | 1.7896 | 0.4102 | GH |
| 5A | 0.3% | 1.6161 | 0.3852 | H |
| 11 | 0.3% | 1.6026 | 0.3894 | H |
| 25 | 0.2% | 1.2607 | 0.3921 | H |
| 10 | 0.1% | 0.4487 | 0.3714 | I |
| MID | 0.1% | 0 | 0.4288 | IJ |
| 22 | 0.1% | −0.2345 | 0.4289 | IJ |
| LOW | 0.0% | −0.9446 | 0.5624 | J |

*Referring to Table 3: “Overall Probability” refers to the probability that the code will be selected by panelist as being the softest. “Log Odds” refers to the softness value calculated from the ranking data. The higher the log odds, the softer the substrate feels. “95% Grouping” refers to how the substrates can be grouped based on their log odds value because the statistics are similar.

*Codes indicated by “HIGH”, “MID” and “LOW” relate to the control codes for fabric materials used in each study for comparison of feel. The control codes used are indicated in the Table 4 below:

TABLE 4
Control Samples

| Feel | | | |
|---|---|---|---|
| Description | LOW | MID | HIGH |
| Soft | White Crepe Suiting | Satin Tafeta | Blizzard Solid Fleece |

### Example 2

Example 2 is a prophetic feminine hygiene pad of the present invention comprising a modified intake layer fluid permeable bodyside substrate, a fluid impermeable backsheet and an absorbent core disposed therebetween. The modified intake layer is a 40 gsm coform comprising approximately 60% pulp fibers, 40% meltblown fibers and is coated with the benefit agent “A”, Table 1, comprising about 24% HYPOD 8510 polyolefin dispersion available from DOW Chemical, 70% water and 6% silica gel, available from Grace Davidson, Md. The benefit agent add-on is about 20% weight/weight percent based on the coform substrate weight. The fluid permeable modified intake layer is cut to the desired pad dimensions and shape, such as hourglass shape, and is adhered in the periphery to a fluid impermeable baffle such a 1.0 mil XP-3473A White polyethylene film available from Pliant of Schaumburg, Ill. using, for example, adhesive and/or ultrasonic bonding methods. The absorbent core, enveloped between the fluid permeable modified intake layer and the baffle, is a 200 gsm airlaid composite available from Concert Industries, comprising approximately 77% Koch Cellulose Golden Isles 4881, pulp fibers, 8% bicomponent polyethylene/polypropylene binder fibers, and 15% superabsorbent polymer (from BASF) SAP. The feminine pad is provided with other functional features such as attachment mechanism e.g. adhesive peel strip on the garment side of the baffle substrate for attaching the pad to User's underwear.

### Example 3

Example 3 is another prophetic example and is the same as example 2, except the fluid permeable bodyside (modified intake layer) substrate is a coform material with a basis weight of about 200 gsm.

### Example 4

Example 4 is another prophetic example and is the same as examples 2 or 3, except that the fluid permeable bodyside (modified intake layer) substrate is provided with a plurality of apertures. Aperture diameters may range from about 0.1 mm to about 5 mm, from about 0.4 mm to about 1.5 mm or from about 0.5 to about 1.0 mm. The aperture density may also range from about 7.8×10³ holes/m2 to about 1.9×10⁶ holes/m², from about 4.7×10⁴ holes/m2 to about 9.3×105 holes/m2 or from about 1.6×10⁵ holes/m2 to about 3.1×10⁵ holes/m2.

### Example 5

Example 5 is another prophetic example and is the same as example 2, except the fluid permeable bodyside (modified intake layer) substrate is an airlaid substrate such as a 125 gsm Thermal Bonded Airlaid Web comprising about 81 percent pulp fibers and 19% bicomponent (PE/PP) binder fibers.

### Example 6

Example 5 is another prophetic example and is the same as examples 2, 3 or 5, except the benefit agent “B”, comprising about 24% HYPOD 8510 from Dow Chemical, 6% Syloid 244 FP Silica Gel from Grace Davidson, Columbia Md., 65% water and 5% Lutensol A65N, non-ionic surfactant from BASF, is used to coat the modified intake layer substrate material wherein said coating weight add-on is in the range of about 5% to 20% of the substrate weight.

### Example 7

Example 7 is another prophetic example and is the same as examples 1-6 except the benefit agent add-on is about 10% weight/weight percent by weight of the substrate (based on the basis weight of the substrate).

### Example 8

Example 8 is another prophetic example and is the same as example 7 except the benefit agent add-on is about 5% weight/weight percent by weight of the substrate (based on the basis weight of the substrate).

### Example 9

Example 9 is a prophetic feminine hygiene pad of the present invention comprising a modified intake layer (modified intake layer) fluid permeable bodyside nonwoven substrate, such as in Table 2, with a basis weight ranging from about 14 gsm to 200 gsm, a fluid impermeable backsheet and an absorbent core disposed therebetween. A most preferred modified intake layer is a 20 gsm-150 gsm Through-Air Bonded Carded Web (TABCW) coated with a benefit agent selected from “A, B, C, D, E or F”, Table 1, with agent add-on amount in the range of about 3% to 20% weight/weight percent by weight of the substrate (based on the basis weight of the substrate). The fluid permeable modified intake layer is cut to the desired pad dimensions and shape, such as hourglass shape, and is adhered in the periphery to a fluid impermeable baffle such a 1.0 mil XP-3473A White polyethylene film available from Pliant of Schaumburg, Ill. using, for example, adhesive and/or ultrasonic bonding methods. The absorbent core, enveloped between the fluid permeable modified intake layer and the baffle, is a 200 gsm airlaid composite available from Concert Industries, comprising approximately 77 Koch Cellulose Golden Isles 4881, pulp fibers, 8% bicomponent polyethylene/polypropylene binder fibers, and 15% superabsorbent polymer (from BASF) SAP. The feminine pad is provided with other functional features such as attachment mechanism e.g. adhesive peel strip on the garment side of the baffle substrate for attaching the pad to User's underwear.

### Example 10

Example 10 is a prophetic feminine hygiene pad of the present invention comprising a modified intake layer fluid permeable bodyside nonwoven substrate, such as in Table 2, with a basis weight ranging from about 14 gsm to 200 gsm, a fluid impermeable backsheet and an absorbent core disposed therebetween. A preferred modified intake layer is a 20 gsm-150 gsm spunlace (hydroentangled) nonwoven, comprising for example, 50% pulp and 50% synthetic fibers and coated with a benefit agent selected from “A, B, C, D, E or F”, Table 1, with said agent add-on amount in the range of about 3% to 20% weight/weight percent by weight of the substrate (based on the basis weight of the substrate). The fluid permeable modified intake layer is cut to the desired pad dimensions and shape, such as hourglass shape, and is adhered in the periphery to a fluid impermeable baffle such a 1.0 mil XP-3473A white polyethylene film available from Pliant of Schaumburg, Ill. using, for example, adhesive and/or ultrasonic bonding methods. The absorbent core, enveloped between the fluid permeable modified intake layer and the baffle, is a 200 gsm airlaid composite available from Concert Industries, comprising approximately 77 Koch Cellulose Golden Isles 4881, pulp fibers, 8% bicomponent polyethylene/polypropylene binder fibers, and 15% superabsorbent polymer (from BASF)SAP. The feminine pad is provided with other functional features such as attachment mechanism e.g. adhesive peel strip on the garment side of the baffle substrate for attaching the pad to User's underwear.

### Example 11

Example 11 is another prophetic example and is the same as examples 9 and 10, except the absorbent core composition comprises fluff pulp in the range of 0-100% and superabsorbent polymers in the arrange of 0-100%.

### Example 12

Example 12 is another prophetic example and is the same as examples 11, except the prophetic personal care article is a diaper instead of a feminine hygiene product; and the preferred substrate is a nonwoven with a basis weight in the range of about 10 gsm to about 150 gsm, said nonwoven is selected from spunbond and nonwoven laminates such as spunbond-meltblown-spunbond (SMS).

### Example 13

Example 13 is another prophetic example and provides a plurality of apertures to the modified intake layer of the above examples, wherein aperture diameters may range from about 0.1 mm to about 5 mm, from about 0.4 mm to about 1.5 mm or from about 0.5 to about 1.0 mm. The aperture density may also range from about 7.8×10³ holes/m² to about 1.9×10⁶ holes/m2, from about 4.7×10⁴ holes/m² to about 9.3×10⁵ holes/m2 or from about 1.6×10⁵ holes/m2 to about 3.1×10⁵ holes/m². The apertures may extend through the modified intake layer from the external, body-facing surface through and to the internal surface of the modified intake layer.

### Example 14

Example 14 is another prophetic example that provides calendering (embossing) of different designs, shapes and patterns to the modified intake layer of the personal care article such as in examples, 9, 10, 11, 12 and 13, to enhance visual aesthetics and appeal to User.

### Example 15

Example 15 is a step-by-step description of a process example, for applying the benefit agent composition to the substrates of the present invention.
1. Nonwoven substrate (TABCW, Spunlace, Coform, airlaid, spunbond, SMS etc.) is unwound.
2. Dispersion (preferred) or solution of benefit agent, comprising e.g. HYPOD8510 or 8102.2, additives (e.g. Lutensol A65 N, HPC, cotton linters, silica gel, Unifroth 0154) and water is froth foamed onto a heated calender roll (also referred to as dryer, heated drum etc.).
3. Heated drum (crepe drum, cast iron drum) heats the solution and evaporates the water while melting the solids in the solution/dispersion into a thin film-coating on the heated drum.
4. Nip roll applies pressure to adhere film to the nonwoven substrate.
5. Creping/skimming blade scrapes film/nonwoven substrate off drum to produce form a coated substrate such as the modified intake layer of the present invention.
6. The aperture unit pin apertures coated substrate to provide fluid passage pathways through the fluid permeable bodyside substrate e.g. modified intake layer.
7. Embossing/calendering roll applies calendering patterns and designs the fluid permeable bodyside substrate.
8. The substrate material coated with the benefit agent is wound up on winder and shipped to converting asset for manufacturing the personal care article of the present invention.

### Example 16

In example 16, a nonwoven substrate, e.g. spunbond, of relatively low basis weight (e.g. 20 gsm or less) is first coated on its external (garment-facing) surface with the benefit agent of the present invention as described in example 15, for example. The internal surface is then laminated to a fluid impermeable substrate such as a baffle of example 2 film to form a nonwoven/film laminate for use as the fluid impermeable garment side substrate of the present invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care absorbent article comprising at least two substrates each having an internal and external surface,
wherein at least one substrate is a fluid permeable bodyside substrate selected from spunbond, meltblown, coform, airlaid, bonded-carded web, spunlace materials and combinations thereof;
at least one substrate is an impermeable backsheet; and
an absorbent core disposed in between said substrates;
wherein at least the external surface of at least one substrate has applied to it a benefit agent selected from an additive composition wherein said additive composition is a polymer dispersion selected from polyolefin dispersions, polyisoprene dispersions, polybutadiene-styrene block copolymer dispersions, latex dispersions, polyvinyl pyrrolidone-styrene copolymer dispersions, polyvinyl alcohol-ethylene copolymer dispersions, and combinations thereof; an enhancement component selected from microparticles, expandable microspheres, fibers, additional polymer dispersions, scents, anti-bacterials, moisturizers, medicaments, soothers and combinations thereof; and combinations thereof.

2. The personal care article of claim 1 wherein said fluid permeable bodyside substrate has a basis weight from about 5 gsm to about 200 gsm.

3. The personal care article of claim 1 wherein the benefit agent is frothed and bonded to the external surface of at least one substrate through a creping process.

4. The personal care absorbent article of claim 1 wherein the external surface of at least one substrate is applied with from about 3% to about 20%, by weight of the substrate, of a benefit agent.

5. The personal care absorbent article of claim 1 wherein the additive composition may be applied onto the external surface of at least one substrate at a level from about 50 mg/m² to about 50,000 mg/m².

6. The personal care article of claim 1 wherein the benefit agent is an additive composition comprising from about 10% to about 32%, by weight of the benefit agent, a polyolefin dispersion.

7. The personal care absorbent article of claim 1 wherein the benefit agent is an enhancement component.

8. The personal care absorbent article of claim 6 wherein the benefit agent is a combination further comprising an enhancement component that is added to the polymer dispersion in an amount from about 0.5% to about 30%, by weight of the benefit agent.

9. The personal care absorbent article of claim 1 wherein the additive composition is a water-insoluble polyolefin copolymer selected from ethylene-acrylic acid, polyethylene-octene copolymer, and combinations thereof.

10. The personal care absorbent article of claim 1 wherein the additive composition is selected from a synthetic water-soluble polymer, a natural water-soluble polymer and mixtures thereof.

11. The personal care article of claim 1 wherein said fluid permeable bodyside substrate has a saline or menses simulant fluid absorbent capacity greater than from about 1 g/g to about 10 g/g.

12. The personal care article of claim 1 wherein said fluid permeable bodyside substrate is provided with a plurality of apertures extending from said external surface through said internal surface for fluid passage to said absorbent core

13. The personal care article of claim 1 wherein said article is a product selected from a feminine hygiene product, a diaper, a training pant, and an adult incontinence product.

14. The personal care article of claim 7 wherein said article is a product selected from a feminine hygiene product, a diaper, a training pant, and an adult incontinence product.

15. The personal care article of claim 11 wherein said article is a product selected from a feminine hygiene product, a diaper, a training pant, and an adult incontinence product.

16. The personal care article of claim 1 wherein the benefit agent does not leave or transfer from the fluid permeable bodyside substrate to a user.
